(19) United States
(12) Patent Application Publication
(10) Pub. No.: US 2010/0297739 A1
(43) Pub. Date: Nov. 25, 2010

(54) RENEWABLE ENERGY SYSTEM

(75) Inventors: Carl A. Steiner, Erie, PA (US); Ricky L. Jackson, Union City, PA (US)

Correspondence Address: THE WEBB LAW FIRM, P.C., 700 KOPPERS BUILDING, 436 SEVENTH AVENUE, PITTSBURGH, PA, 15219, US

(73) Assignee: TM INDUSTRIAL SUPPLY, INC., Erie, PA (US)

(21) Appl. No.: 12/783,129

(22) Filed: May 19, 2010

Related U.S. Application Data
(60) Provisional application No. 61/180,267, filed on May 21, 2009.

(51) Int. Cl. C12M 3/00; C12N 1/12

(52) U.S. Cl. 435/257.1; 435/292.1

(57) ABSTRACT

An apparatus for cultivating photosynthetic organisms is provided. The apparatus can be used in a renewable energy system. The apparatus comprises a chamber for growing photosynthetic organisms in a liquid medium; a device for holding the photosynthetic organism; a carbon dioxide source; a medium intake source; and a system for providing movement of the liquid medium within the chamber. A system for cultivating photosynthetic organisms comprises a plurality of apparatuses for cultivating photosynthetic organisms, wherein the plurality of apparatuses are interconnected in parallel, series, or a combination thereof, in order to increase algae production. Also described is a method of cultivating a photosynthetic organism.

### CROSS REFERENCE TO RELATED APPLICATIONS

This application claims priority from U.S. Provisional Application No. 61/180,267, filed May 21, 2009, which is incorporated herein by reference.

### BACKGROUND OF THE INVENTION

1. Field of the Invention

This invention relates to an apparatus for cultivating photosynthetic organisms. The apparatus can be used in a renewable energy system, such as a system which uses biomass to produce fuels, e.g., ethanol and bio-diesel; chemicals, e.g., glycerin; and dry distilled grains (DDGs) for feeding livestock, e.g., cows. A multitude of these apparatuses for cultivating photosynthetic organisms may be interconnected in parallel, series, or a combination thereof in order to increase algae production. The invention also relates to a process for cultivating photosynthetic organisms in a renewable energy system.

2. Description of Related Art

Renewable energy processes may involve one or more renewable energy resources, such as solar, wind, water, plants, animals, and municipal wastes, since these resources always exist. Renewable energy resources generally offer clean alternatives to fossil fuels, e.g., coal, oil and natural gas, for supplying most energy needs, since the renewable energy resources produce very little or no pollution or greenhouse gases.

A prior art renewable energy process uses coal bed methane, coal gasification, and landfill gas recovery for biogas production used to produce steam, heat, and electricity needed in the cycle. Manure slurries are used to produce dry fertilizer products for fertilizing the corn fields. The corn is used in the production of ethanol and bio-diesel. Such a process is illustrated in the brochure entitled “Where Can Fluid Engineering Help Your Bio-fuel Plant?” provided by Fluid Engineering, Erie, Pa.

Among many things, this illustration shows that carbon dioxide produced within the system can be bubbled into ponds to grow algae that can be converted into bio-diesel fuel, and one source of carbon dioxide is from the fermentation tanks for the corn. Corn is used to produce DDGs for feeding livestock. Corn, corn oil, algae, and other products are used to produce bio-diesel which is made through a chemical process called transesterification, whereby glycerin is separated from the fat and/or vegetable oils. The transesterification process leaves behind two products—methyl esters, which is the chemical name for bio-diesel, and glycerin, which is used in food and beverages, pharmaceuticals, cosmetics and toiletries, paper and printing, textiles, livestock, and biodegradable packaging.

It can be appreciated that this prior art renewable energy process involves a complicated system and uses coal bed methane, coal gasification, landfill gas recovery and manure slurries as its main input components for producing outputs such as ethanol and bio-diesel.

Algae culture is often done in ponds and lakes which are open to the elements. This type of culturing is vulnerable to contamination by microorganisms, such as bacteria. Open pond systems do not allow control over water temperature and lighting conditions. In addition, the growing season is largely dependent on location and is limited to the warmer months. These factors limit the number of species successfully cultivated in an open-pond system. However, open-pond systems are often less expensive to operate and offer large growth areas.

Algae may also be grown in closed systems, often referred to as a photobioreactor. A photobioreactor incorporates some type of light source. These structures are generally smaller systems, and, for economic reasons, often do not solve many of the problems associated with an open system.

One advantage to utilizing algae in a renewable energy system is that this organism can grow in salt water, freshwater, or even contaminated water. In addition, algae show accelerated growth in the presence of excess carbon dioxide (the main greenhouse gas) and organic material like sewage. Algae may be used to clean carbon dioxide or untreated sewage. The oil produced by algae can then be harvested and converted into bio-diesel and the algae's carbohydrate content can be further fermented into ethanol. Both of these substances are much cleaner burning fuels than petroleum-based diesel or gas.

One impediment to the expansion of large scale algae culture for pollution abatement has been the difficulty in developing an efficient and cost effective culture system. Growth in large open environments makes control of the temperature, nutrient, carbon dioxide and light levels difficult. Thus, a need exists for the controlled optimization of growth conditions in a closed culture environment.

### SUMMARY OF THE INVENTION

According to one aspect, the invention is directed to an apparatus for cultivating photosynthetic organisms, comprising: a chamber for growing photosynthetic organisms in a liquid medium; a device for holding the photosynthetic organism; a carbon dioxide source; a medium intake source; and a system for providing movement of the liquid medium within the chamber. According to one embodiment, the apparatus is constructed of a material that is at least partially transparent to light or configured to transmit or reflect light, such as plastic, glass, fiberglass, alloy steel and aluminum. The surface of the apparatus can be lined with a light reflective material, such as aluminum or alloy steel. The chamber can be enclosed or partially enclosed and can be radiused. The light source can comprise an artificial light source or can be a combination of an artificial light source and sunlight. The artificial light source can comprise Fresmel-lense-based lenses, a Himanari solar concentration and transmission system, and a mirror based optical wave guide solar lighting system. The liquid medium movement system can comprise a paddle wheel. The carbon dioxide can be provided from a power generation system or an engine system and the medium intake source can comprise a water source and a nutrient source. The nutrient source can be provided from agricultural waste or municipal sewage. The apparatus can include one or more monitors to measure temperature, pH, carbon dioxide, light intensity, and oxygen levels in the liquid medium. The device for holding the photosynthetic organism can comprise a perforated plate barricade located within the chamber which is in contact with the moving liquid medium for holding the photosynthetic organism in a fixed position. The apparatus can further include at least one of a gas sparger for introducing gas or air bubbles into the liquid medium and an agitation member to enhance movement of the liquid medium. The apparatus can also include a liquid medium outtake for removal of liquid medium used for the culture of the photosynthetic organism. A heat source can be provided for maintaining a predetermined temperature within the system based upon the photosynthetic organism culture being grown.

According to another aspect, the invention is directed to a system for cultivating photosynthetic organisms comprising a plurality of apparatuses. Each of the apparatuses comprises a chamber for growing photosynthetic organisms in a liquid medium; a device for holding the photosynthetic organism; a carbon dioxide source; a medium intake source; and a system for providing movement of the liquid medium within the chamber. The plurality of apparatuses can be interconnected in parallel, in series, or in a combination thereof. The plurality of apparatuses can be stacked in a multi-level design which allows for the simultaneous growth culture of different photosynthetic organisms. The apparatuses can have separate light sources, medium supplies and carbon dioxide supplies. According to another design, the apparatuses can share common medium and carbon dioxide supplies. The apparatuses can be movable with respect to one another and/or to change the angular orientation thereof.

According to yet another aspect, the invention is directed to a method of cultivating a photosynthetic organism, comprising the steps of providing the photosynthetic organism to an apparatus; feeding a medium into the apparatus; moving the medium through the apparatus; adding nutrients and carbon dioxide to the medium; and providing a light source to the apparatus. The apparatus for receiving the photosynthetic organism can be formed from a material that is at least partially transparent to light or configured to transmit or reflect light. The light source can comprise an artificial light source, sunlight, or a combination of both. The method can further include the step of agitating the liquid medium.

These and other features and characteristics of the present invention, as well as the methods of operation and functions of the related elements of structures and the combination of parts and economies of manufacture, will become more apparent upon consideration of the following description with reference to the accompanying drawings, all of which form a part of this specification.

### BRIEF DESCRIPTION OF THE DRAWINGS

FIG. 1 is a schematic illustrating a renewable energy system of the present invention.

FIG. 2 is a schematic illustrating an additional embodiment of the renewable energy system of the present invention.

FIG. 3 is a schematic illustrating an additional embodiment of the renewable energy system of the present invention.

FIG. 4 is a schematic illustrating an additional embodiment of the renewable energy system of the present invention.

FIG. 5A is a top view illustrating an algae bed of the present invention.

FIG. 5B is a perspective view illustrating the additional embodiment of the algae bed of the present invention.

FIG. 6A is a perspective view illustrating an algae bed system of the present invention.

FIG. 6B is a side view illustrating the additional embodiment of the algae bed of FIG. 6A.

FIG. 6C is another side view illustrating the additional embodiment of the algae bed of the present invention.

FIG. 7A is a top view illustrating an additional embodiment of an algae bed of the present invention; and

FIG. 7B is a perspective view illustrating the additional embodiment of the algae bed of FIG. 7A.

### BRIEF DESCRIPTION OF THE INVENTION

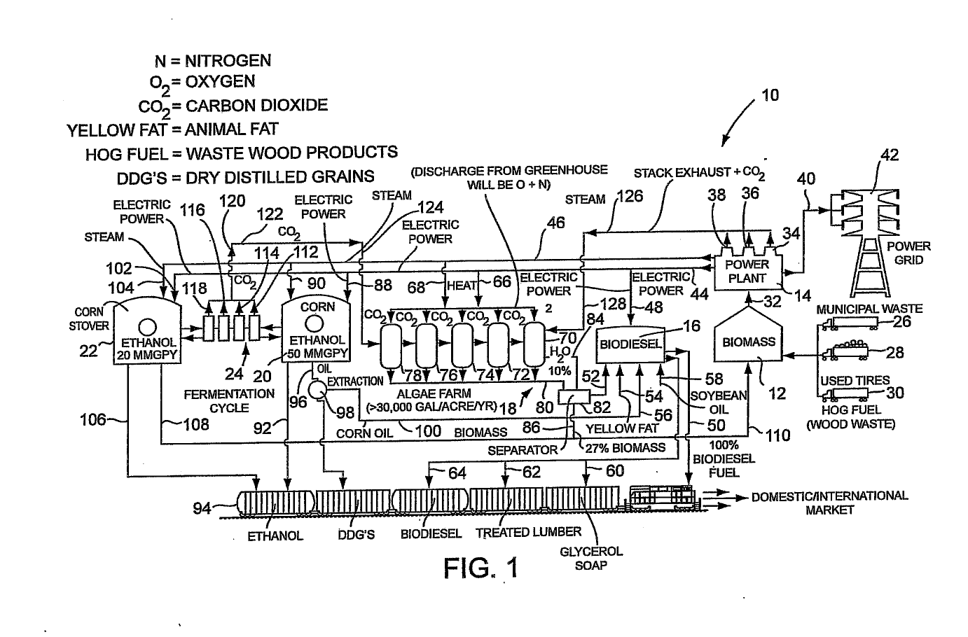

A renewable energy system 10 of FIG. 1 includes a biomass unit 12, a power plant 14, a bio-diesel unit 16, an algae farm 18, a corn unit 20, a corn stover unit 22, and a fermentation cycle unit 24 associated with the corn unit 20, and the corn stover unit 22. The biomass unit 12 is supplied with biomass energy sources, such as organic components from municipal wastes, as indicated by reference number 26, rubber from used tires, as indicated by reference number 28, and hog fuel, also known as wood waste, which includes agricultural forestry waste and residue, as indicated by reference number 30. Other non-limiting alternative sources of biomass energy may include wood, food crops, e.g., cornhusks, grasses and other plants, organic components from industrial wastes, sewage, and methane gas harvested from community landfills. A person of skill in the art would recognize that there are numerous sources of biomass energy. As alluded to hereinabove, biomass is a renewable energy source since trees and crops will continue to grow and waste will continue to exist. This material generally is known to contain residual energy, which can be released by burning it in biomass power plants, such as that indicated by reference number 12 in FIG. 1.

As shown by arrow 32 in FIG. 1, the biomass in biomass unit 12 is fed to the power plant 14 which produces electricity and steam, and which discharges exhaust solids and carbon dioxide (CO₂) from stacks 34, 36 and 38. As indicated by an arrow 40, to the right of power plant 14, electricity is provided to a power grid 42 for human use. As indicated by an arrow 44, to the left of power plant 14, electric power is delivered to the several units and, as indicated by an arrow 46, to the left of power plant 14, steam is delivered to the several units of system 10 for their operation.

As shown by an arrow 48, electric power is delivered to the bio-diesel unit 16 to produce bio-diesel fuel, as indicated by an arrow 50, which can be used directly in any type of diesel engine or can be shipped to domestic and international markets for human use. As shown, the bio-diesel fuel is produced from algae oil, as indicated by an arrow 52, animal fat or yellow fat, as indicated by an arrow 54, corn oil, as indicated by an arrow 56, and soybean oil, as indicated by an arrow 58. As discussed hereinabove, bio-diesel fuel is made through a chemical process referred to as transesterification, whereby glycerin is separated from the fat and vegetable oils. The glycerin may be used in the manufacture of glycerol soap as shown by an arrow 60, used to treat lumber, as shown by an arrow 62, and is used as bio-diesel fuel, as shown by an arrow 64. One of skill in the art would recognize that the glycerin produced by this process may have multiple applications. Bio-diesel fuel has advantages over diesel fuel which is made from fossil fuels, e.g., coal and natural gas, in that it burns cleaner, is renewable, and does not cause air pollution. Also, bio-diesel fuel can be made from cooking oil and other types of fresh oils.

To the left of the bio-diesel unit 16 of FIG. 1, and as indicated respectively by arrows 66 and 68, electric power and steam in the form of heat are delivered or supplied from power plant 14 to algae pond units 70, 72, 74, 76 and 78 of algae farm 18. As indicated, algae farm 18 produces more than 30,000 gallons of algae per acre per year. It has recently become appreciated that algae are the ultimate feedstock for producing bio-diesel fuel. As indicated by an arrow 80, these algae are delivered to a separator 82 to produce three outputs, which are oil, water, and biomass. Approximately 63% is algae oil, which is fed, as indicated by arrow 52, into bio-diesel unit 16; about 10% is water; and about 27% is biomass. As indicated by an arrow 84, the water from separator 82 is cycled into the algae pond units, and the biomass, as indicated by an arrow 86, along with the biomass from the corn stover unit 22, is fed back to the biomass unit 12, about which more will be discussed hereinbelow.

To the left of algae farm 18 of FIG. 1, and as indicated respectively by arrows 88 and 90, electric power and steam from power plant 14 is delivered to corn unit 20 which are used to produce ethanol and DDGs. This corn unit 20 could produce approximately 50 million gallons of ethanol per year. The amount of ethanol produced is a function of the size of the corn unit 20 and the amount of corn supplied thereto. In one non-limiting embodiment of the invention, as indicated by an arrow 92, the ethanol is delivered to a railroad car 94 which is then shipped for human use. One of skill in the art will recognize that the ethanol can be delivered to and distributed by any acceptable transport system. As indicated by an arrow 96, the DDGs are subjected to an oil extraction device 98, whereby corn oil is extracted from the DDGs. As indicated by arrow 100, and as discussed hereinabove, this corn oil is delivered to the bio-diesel unit 16.

To the left of corn unit 20, and as indicated respectively by arrows 102 and 104, electric power and steam from power plant 14 is delivered to corn stover unit 22, which are used to produce ethanol and biomass, as indicated by arrows 106 and 108, respectively. This corn stover unit 22 produces approximately 20 million gallons of ethanol per year. In one non-limiting embodiment, as indicated by an arrow 106, the ethanol is also delivered to railroad car 94, which is then shipped for human use. As is known, corn stover is a by-product of corn and is made from stalks or husks that remain once the corn is harvested. As indicated by an arrow 108, the cornhusks and stalks, now considered as sources of biomass energy, are delivered, as indicated by an arrow 110, to the biomass unit 12. The amount of ethanol produced is a function of the size of the corn stover unit 22 and amount of corn stover supplied thereto.

The fermentation cycle unit 24 is located between and is associated with corn unit 20 and the corn stover unit 22 in FIG. 1. The fermentation unit 24 ferments the corn and the corn stover in the production of ethanol. As indicated by the upward arrows 112, 114, 116 and 118, carbon dioxide is discharged from this fermentation unit 24. As indicated by arrows 120, 122 and 124 leading out of the fermentation unit 24, this carbon dioxide gas is delivered to the algae farm 18, as indicated to the left of algae farm 18. As indicated by arrows 126 and 128 to the right of algae farm 18, and leading out of the exhaust stacks 34, 36 and 38 of power plant 14, the carbon dioxide gas produced from power plant 14 is delivered to the algae farm 18. In a known manner, this carbon dioxide gas is bubbled into the algae ponds 70, 72, 74, 76 and 78 to grow the algae that then are converted into bio-diesel fuel and a discharge of oxygen and oxygen byproducts.

An additional aspect of the renewable energy system of the invention involves the use of algae as a bio-fuel. The invention contemplates the practice of algaculture (farming algae) for making vegetable oil, bio-diesel, and other biofuels. However, it should be understood that the present invention is not limited to the production of algae, but may be applicable to the culture of other photosynthetic organisms. The term “photosynthetic organism”, as used herein, includes any organism capable of photosynthetic growth. These terms may be used to include organisms modified artificially or by gene manipulation.

The inventive system of the invention utilizes algae to treat agricultural waste or sewage to remove toxins. The system can be used to remove pollutants emitted into the atmosphere from a facility and to produce biomass that can then be converted into non-fossil fuels. This system can be used for treating wastes produced by treatment plants, agricultural centers, or other such facilities. The algae or other photosynthetic organisms can utilize the carbon dioxide from these facilities for growth while producing biomass.

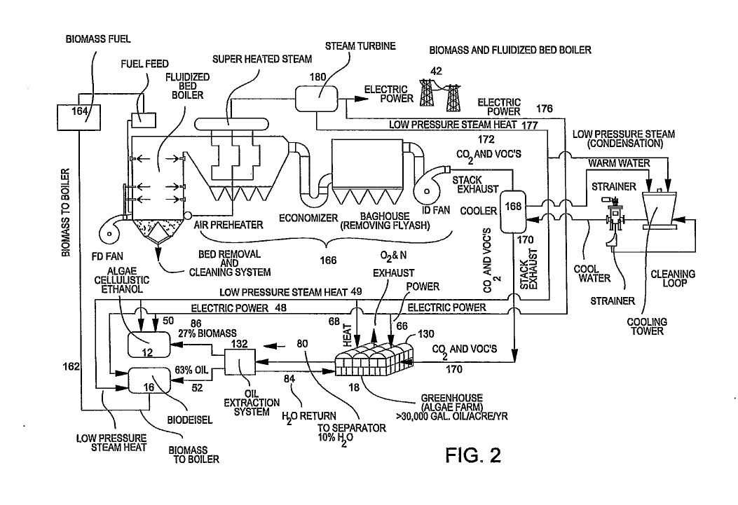

In one aspect, illustrated in FIGS. 2-4, the present invention comprises growing algae in an enclosed system or chamber (not exposed to open air). This avoids the problem of contamination by other organisms present in the open environment. The closed system requires a source of carbon dioxide. The carbon dioxide from a smokestack may be used for growing algae. In order to minimize expense, the algae farming may be located in close proximity next to a power generating plant. In this aspect, the algae farm may be used to reduce pollution produced by the power generating plant.

In an alternative aspect, the growing chamber may be partially enclosed, thus allowing the access of environmental air, nutrients, and/or light.

In an additional aspect of the invention, one nutrient source for the algae farm is waste water from the treatment of sewage, agricultural power generation, or flood plain run-off, all currently major pollutants and health risks. It is useful to first treat this waste water by processing with bacteria, through anaerobic digestion. Processing of the waste water prevents contamination of the algae. The organic waste is converted to a mixture of carbon dioxide, methane, and organic fertilizer. Organic fertilizer, which exits the digester as a liquid, may first be cleaned and sterilized. Growth of the algae can be increased and accelerated when fed extra carbon dioxide (the main greenhouse gas) and organic material like sewage.

FIG. 2 illustrates one embodiment of the invention utilizing carbon dioxide and other waste products for the production of algae. Here, as indicated by arrow 176, electric power is supplied from power grid 42 and directed to algae farm 18 (arrows 176 and 66). Heat in the form of steam is supplied from steam turbine 180 to the algae farm 18 (see arrow 177). Carbon dioxide is provided from power generation system 166 to cooler 168 (arrow 172) and then to algae farm 18 as indicated by arrow 170. Harvested algae are then transferred (arrow 80) to an oil extraction system 132. Here, the algae is processed and separated into (a) algae oil, which is transferred to a bio-diesel generator 16 (arrow 52) for production into bio-diesel fuel 164 (arrow 162), and (b) biomass, which is transferred (arrow 86) to a biomass unit 12.

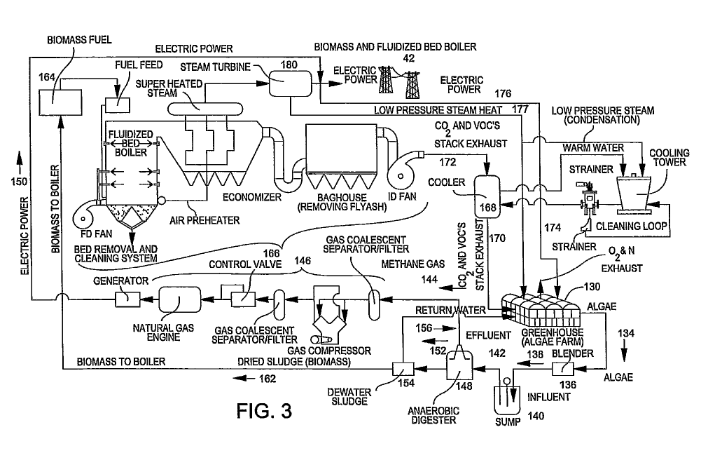

In an additional aspect, as illustrated in FIG. 3, electric power is supplied from power grid 42 and directed to algae farm 18 (arrows 174 and 176). Heat in the form of steam is supplied from steam turbine 180 and power generation system 166 to the algae farm 18 (see arrow 177). Carbon dioxide is provided to algae farm 18 from the power generation system 166, as indicated by arrow 170. Here, as indicated by arrow 134, the harvested algae is provided to a blender 136 and then, as indicated by arrow 138, moved to a sump 140. The algae are then moved, as represented by arrow 142, to anaerobic digester 148. From here, the algae is then processed to dewater sludge 154 (arrow 152) and then processed to biomass fuel 164. The methane gas produced by digester 148, as indicated by arrow 144, may then be used for the operation of power electric plant (arrow 150).

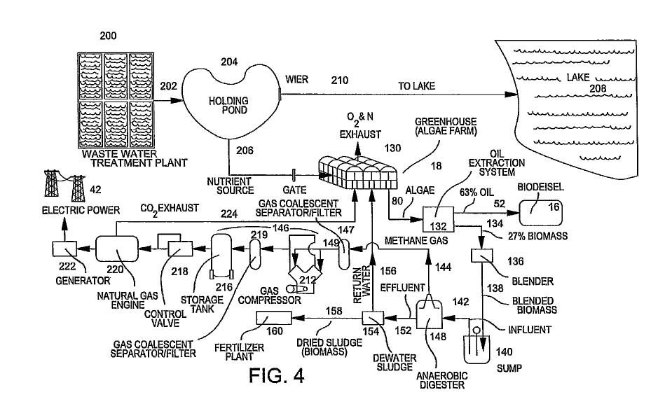

FIG. 4 illustrates a further embodiment of the invention. Here, a waste water treatment plant 200 provides waste water, illustrated by arrow 202, to a holding pond 204. Holding pond 204 is used as a nutrient source, as represented by arrow 206, to greenhouse 130 and algae farm 18. Purified overflow from holding pond 204 may also be directed to a lake 208 or other body of water, as indicated by arrow 210. Harvested algae are then directed to an oil extraction system 138 (represented by arrow 80). Here, the algae is processed and separated into (a) algae oil, which is transferred to a bio-diesel generator 16 (arrow 52) for production into bio-diesel fuel, and (b) biomass, which is transferred (arrow 134) to a blender 136. The biomass is then moved, as represented by arrow 142, to anaerobic digester 148. From here, the algae is processed to dewater sludge 154 (arrow 152) and then transferred, as indicated by arrow 158, to a fertilizer plant 160. The methane gas produced by anaerobic digester 148, as indicated by arrow 144, is moved through a natural gas coalescent/generation system 146 to be processed for use in the operation of power electric plant 42. Here, the methane travels through a gas coalescent separator/filter 147 to gas compressor 212 (arrow 149) then to a second gas coalescent separator/filter 219. The processed gas is then processed through storage tank 216, control valve 218 and natural gas engine 220 before powering generator 222. In addition, return water from the dewater sludge 154 may be returned to the greenhouse 130, as represented by arrow 156. Carbon dioxide gas from natural gas engine 220 may then be directed to greenhouse 130 for the production of algae or other photosynthetic organisms, as represented by arrow 224.

The greenhouse 130 and algae farm 18 will now be described in more detail. In order to grow and maintain a pure algae culture, control outdoor parameters and extend growing parameters, the present invention allows for indoor culturing using bioreactors and waste carbon dioxide. The present invention further allows for lipid production for bio-diesel and other compositions, based upon the optimization of outdoor culture parameters, including carbon dioxide utilization.

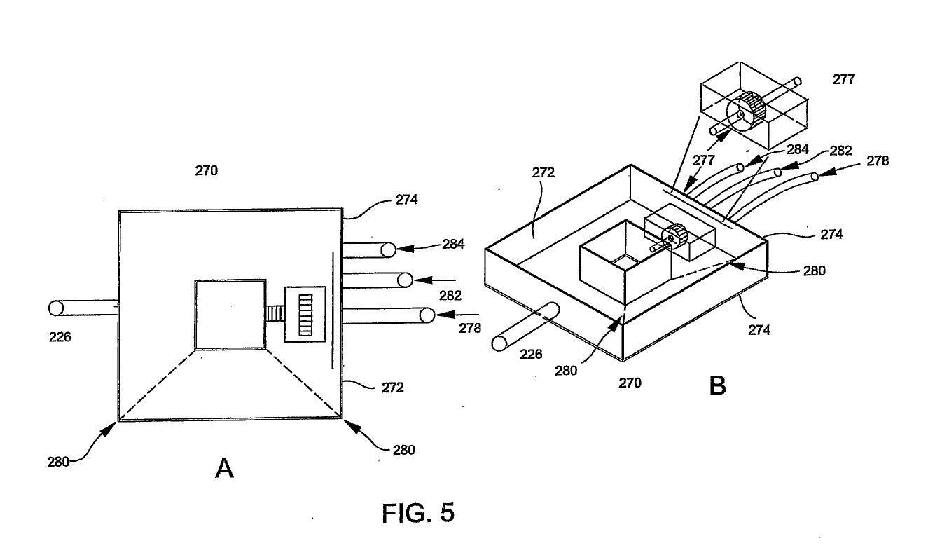

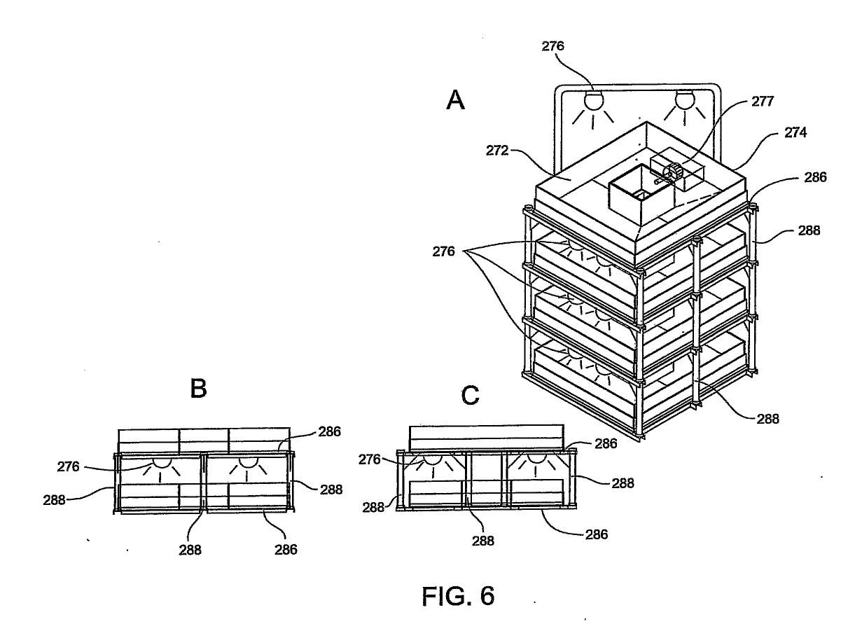

As illustrated in FIGS. 5A, 5B, 6A, 6B and 6C, the current invention contemplates algal culture using a closed race-way system. These systems may be utilized in conditions where the temperature or the amount of sunlight may be less than optimal. The closed system is preferred to maintain a pure culture, prevent water evaporation, maximize space and light intensity, and overcome sunshine limitations due to climate.

FIGS. 5A and 5B illustrate several aspects of the algae bed 270 of the invention. The term “algae bed” refers to an apparatus configured to contain a photosynthetic organism in a liquid medium. Algae bed 270 is constructed with a material 272 at least partially transparent to light or configured to transmit or reflect light. Material 272 must be capable of transmitting or reflecting at least enough light energy to drive photosynthesis within the photosynthetic organism. Suitable materials include, but are not limited to, transparent materials such as plastic, glass, or fiberglass, or reflective materials such as alloy steel. Material 272 must be sufficiently rigid to support the algae bed through normal operation and to withstand outdoor elements and forces. Algae bed 270 is lined on the outside or inside with a light reflective material 274 to reflect light from the outside walls and bottom of bed 270. The light reflective material 274 may comprise any composition that will effectively reflect light from the light source. Non-limiting examples of light reflective material 274 are aluminum and stainless steel.

The term “liquid medium” as used herein, refers to any liquid containing sufficient nutrients to facilitate the growth of the photosynthetic organism. Generally, the liquid medium comprises water with added nutrients. Suitable mediums sufficient to support the growth of photosynthetic organisms are well known in the art.

As indicated in FIGS. 6A-6C, an artificial light source providing light at a wavelength able to drive photosynthesis is provided. Light source 276 may be any artificial light transmission instrument capable of providing light at the proper wavelength and intensity. In one non-limiting example, light source 276 comprises Fresmel-lense-based lenses and a Himanari solar concentration and transmission system. Additionally, light source 276 may contain a mirror based optical waveguide solar lighting system. Light source 276 is capable of operating for a user-defined period of time (e.g., 18 hour cycles). Light source 276 allows for light/dark exposure intervals. These intervals may be manually determined by the user or may be computer controlled. In one embodiment, light source 276 utilizes an orbit compact fluorescent light with 576 watt (6-96 watts×72 inches) lunar lights. The periods of time with no illumination are produced by dual actinic 460 nm bulbs. In one embodiment, light source 276 is mounted directly over the algae growing area. In an additional embodiment, an algae bed may utilize both natural sunlight and an artificial light source 276.

Referring again to FIGS. 5A and 5B, algae bed 270 is filled with enough liquid medium so as to permit a recirculation flow of fluid during operation of the bed. It is not necessary for algae bed 270 to be filled to capacity with the liquid medium. The maximum depth of the medium for the reflection light system rays would be 20 cm (7.8 inches) to allow for proper light absorption and water movement. The sides of the pond need only be 12 inches deep to achieve these parameters.

Algae bed 270 includes a system for providing movement of the liquid medium. In one embodiment, this liquid medium movement system comprises a paddle wheel system 277 which allows for the continuous movement of the liquid medium in the device. Paddle wheel 277 provides variable speed movement of the fluid in which the algae is grown. Paddle wheel 277 also allows for a mixing feature to increase gas-liquid mixing within the bed. The photosynthetic organism is held in a fixed position through the use of a perforated plate barricade 280. Nutrients and fertilizer are fed into the water on the back side of the perforated plate barricade and paddle wheel 277 as indicated by arrow 278. Paddle wheel 277 acts to mix the nutrients with the water as it is circulated throughout algae bed 270.

The present system utilizes supplementation with carbon dioxide to increase algae growth. Typically, algae grown in a pond require seven to nine days to grow from bud to maturity (ready to harvest). The use of carbon dioxide enhances algae growth. The present system, which provides supplemental carbon dioxide, shortens the growth phase to three to four days, thus allowing for an increased number of harvests per year. Algae bed 270 includes the addition of a continuous fresh supply of carbon dioxide (as represented by arrow 282). The carbon dioxide is injected into the apparatus provided by a gas sparger tube 284 and placed in front of the perforated plate barricade 280. A “gas sparger tube” refers to any device configured to introduce gas or air bubbles into the liquid nutrient medium. Suitable gas sparger tubes are well known to those of skill in the art. The carbon dioxide is provided to the algae bed 270 from power generation system 166 or the methane gas engine 220. Movement of the nutrient water by paddle wheel 277 over the carbon dioxide sparger tube 284 provides an enriched nutrient feed for the algae. The carbon dioxide and nutrient feeds may further comprise agitation and tubular gas flow systems, containing bubble columns, air lines, and flat panel air lifts with baffles (now shown) to ensure proper movement of the water. Algae bed 270 also contains a liquid medium outtake 226. This allows for the removal and replacement of the liquid medium used for the culture of the photosynthetic organism.

It is important to control the liquid medium temperature in order to maximize photosynthetic organism culture. The specific temperature requirements will depend on the species of organism in culture. For the culture of algae, the nutrient water should be maintained at a temperature from about 10° C. to about 40° C. In the present invention, the heat utilized to maintain the water temperature is provided in the form of low pressure steam heat from the steam turbine 180 and power generation system 166, such as shown in FIG. 3. The temperature may be further regulated by fully submersible heaters (not shown). These additional heaters may be manually adjusted, or they may be controlled through the use of a computer. As will be appreciated by one of skill in the art, any suitable liquid heating system may be utilized in the present invention.

Algae bed 270 may optionally contain various probes and monitors for the measurement of temperature, pH, carbon dioxide, oxygen, light intensity, and other environmental factors. The apparatus may also contain instruments to measure liquid and gas flow rates, as well as illumination intensity levels in the medium.

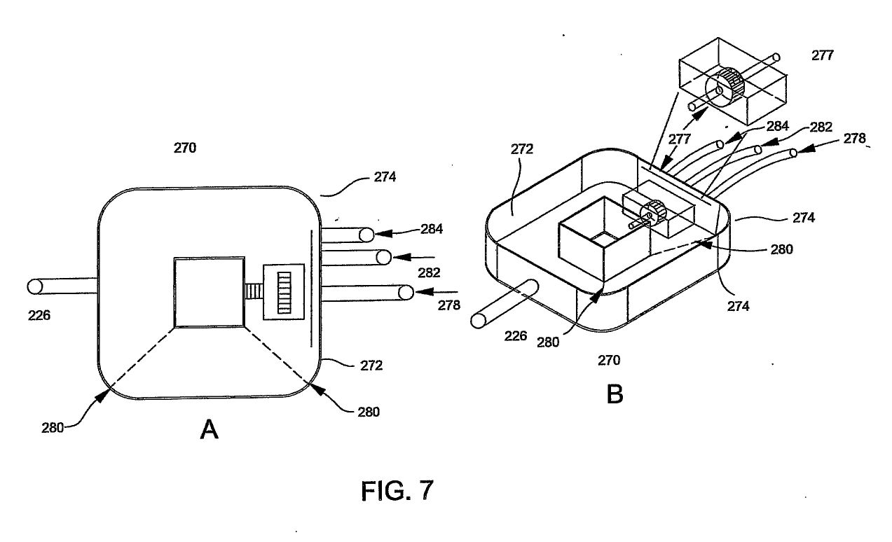

Algae bed 270 may be arranged in a geometry other than the straight angles illustrated in FIGS. 5A, 5B, 6A, 6B and 6C. For example, as illustrated in FIGS. 7A and 7B, algae bed 270 may employ a radiused design to improve fluid dynamics. This design allows for improved flow of the nutrient water. Proper water movement reduces adherence of the algae to the clear plastic material 272 and increases the efficiency of light source 276. As will be clear to one of skill in the art, the exact configuration of the various components of algae bed 270 will depend upon the desired use of the apparatus and the organism to be cultured.

The algae bed apparatus of the present invention can comprise a plurality of identical or similar beds interconnected in parallel, series, or in combination. A parallel configuration of multiple algae beds increases the capacity of the greenhouse 130. In one embodiment, illustrated in FIG. 6A, several algae beds 270 are stacked to create a multi-level design. The stacked design may comprise, in one embodiment, from about 2 to about 8 algae beds 270. The stacked configuration described above may be utilized in the simultaneous growth and culture of different photosynthetic organisms. Different types of organisms, for example, different types of algae having separate lighting and nutritional requirements, may be cultured at different levels of the stackable apparatus. Each level of the stacked apparatus may contain a separate light source 276, system for providing water movement 277, and nutrient/carbon dioxide sources. In another embodiment, the stacked algae beds 270 may share common water, nutrient, and carbon dioxide supplies.

The algae beds of the invention may be positioned by platforms 286 and support posts 288 as illustrated in FIGS. 6B and 6C. Platforms 286 may be stationary. In an alternative embodiment, platforms 286 may be equipped to pivot or turn to change the angle of the algae beds. The changing of the angle of algae beds 270 may be accomplished manually or automatically through the use of a computer.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of this description. For example, it is to be understood that the present invention contemplates that, to the extent possible, one or more features of any embodiment can be combined with one or more features of any other embodiment.

The invention claimed is:

1. An apparatus for cultivating photosynthetic organisms, comprising:
a. a chamber for growing photosynthetic organisms in a liquid medium;
b. a device for holding the photosynthetic organism;
c. a carbon dioxide source;
d. a medium intake source; and
e. a system for providing movement of the liquid medium within the chamber.

2. The apparatus of claim 1, wherein the apparatus is constructed of a material that is at least partially transparent to light or configured to transmit or reflect light.

3. The apparatus of claim 1, wherein the surface of the apparatus is lined with a light reflective material.

4. The apparatus of claim 1, wherein the chamber is radiused.

5. The apparatus of claim 1, wherein the light source comprises an artificial light source.

6. The apparatus of claim 5, wherein the light source comprises:
a. Fresmel-lense-based lenses;
b. a Himanari solar concentration and transmission system; and
c. a mirror based optical wave guide solar lighting system.

7. The apparatus of claim 1, wherein the light source comprises a combination of an artificial light source and sunlight.

8. The apparatus of claim 1, wherein the liquid medium movement system comprises a paddle wheel.

9. The apparatus of claim 1, wherein the carbon dioxide is provided from a power generation system or an engine system.

10. The apparatus of claim 1, wherein the medium intake source comprises a water source and a nutrient source.

11. The apparatus of claim 10, wherein the nutrient source is provided from agricultural waste or municipal sewage.

12. The apparatus of claim 1, further comprising monitors to measure temperature, pH, carbon dioxide, light intensity, and oxygen levels in the liquid medium.

13. The apparatus of claim 1, wherein the device for holding the photosynthetic organism comprises a perforated plate barricade located within the chamber and in contact with the moving liquid medium for holding the photosynthetic organism in a fixed position.

14. The apparatus of claim 1, including at least one of a gas sparger for introducing gas or air bubbles into the liquid medium and an agitation member to enhance movement of the liquid medium.

15. The apparatus of claim 1, wherein the chamber is enclosed or partially enclosed.

16. The apparatus of claim 1, including a liquid medium outtake for removal of liquid medium used for the culture of the photosynthetic organism.

17. The apparatus of claim 1, including a heat source for maintaining a predetermined temperature within the system based upon the photosynthetic organism culture being grown.

18. A system for cultivating photosynthetic organisms comprising a plurality of apparatuses, wherein each apparatus comprises:
a. a chamber for growing photosynthetic organisms in a liquid medium;
b. a device for holding the photosynthetic organism;
c. a carbon dioxide source;
d. a medium intake source; and
e. a system for providing movement of the liquid medium within the chamber; and
wherein the plurality of apparatuses are interconnected in parallel, series, or a combination thereof.

19. The system of claim 18, wherein the apparatuses are stacked in a multi-level design.

20. The system of claim 19, wherein the stacked design allows for the simultaneous growth culture of different photosynthetic organisms.

21. The system of claim 18 wherein the apparatuses share common medium and carbon dioxide supplies.

22. The system of claim 19 wherein the apparatuses are movable with respect to one another and/or to change the angular orientation thereof.

23. A method of cultivating a photosynthetic organism, comprising the steps of:
a) providing the photosynthetic organism to an apparatus;
b) feeding a medium into the apparatus;
c) moving the medium through the apparatus;
d) adding nutrients and carbon dioxide to the medium; and
e) providing a light source to the apparatus.

24. The method of claim 23, wherein the apparatus for receiving the photosynthetic organism is formed from a material that is at least partially transparent to light or configured to transmit or reflect light.

25. The method of claim 23, wherein the light source comprises an artificial light source.

26. The method of claim 23, including the step of agitating the liquid medium.
